(19) United States
(12) Patent Application Publication
(10) Pub. No.: US 2009/0095292 A1
(43) Pub. Date: Apr. 16, 2009

(54) INGESTA ADMINISTRATION DEVICE

(75) Inventors: Soji Hamano, Yokohama-shi (JP); Mitsuru Imai, Chichibu-shi (JP)

Correspondence Address: FITZPATRICK CELLA HARPER & SCINTO, 30 ROCKEFELLER PLAZA, NEW YORK, NY, 10112, US

(73) Assignee: CANON KABUSHIKI KAISHA, Tokyo (JP)

(21) Appl. No.: 12/282,818

(22) PCT Filed: Mar. 30, 2007
(86) PCT No.: PCT/JP2007/057714

§ 371 Date: Sep. 12, 2008

(30) Foreign Application Priority Data
Apr. 10, 2006 (JP) 2006-107138

(51) Int. Cl. A61M 15/00

(52) U.S. Cl. 128/203.14

(57) ABSTRACT

Provided is an ingesta administration device (1) which includes a consumed energy amount obtaining portion (2); an intake energy amount obtaining portion (12); an administration amount determining portion; and an ejection portion (4) for ejecting ingesta such as insulin based on administration amount determined by the administration amount determining portion. The consumed energy amount obtaining portion (2) obtains information on consumed energy amount based on information on exercise amount of the user. The intake energy amount obtaining portion (12) obtains information on amount of energy to be ingested by the user. The determination portion determines administration amount of the ingesta based on at least the information obtained by the consumed energy amount obtaining portion on amount of energy consumed by the user since previous administration until current administration and the information obtained by the intake energy amount obtaining portion on the amount of energy to be ingested after current administration.

### TECHNICAL FIELD

The present invention relates to an ingesta administration device such as a drug ejection device which ejects a drug and allows a user to inhale the drug, and more particularly to a technique for safely using an ingesta administration device such as a drug ejection device for health care which is so configured as to be portably carried by a user.

### BACKGROUND ART

A treatment for a user in which a drug ejection device for allowing the user to intake a drug through inhalation is utilized in combination with information database such as electronic medical records is being embodied.

Such a drug ejection device has a storage means for storing information related to an individual user including information on the medical record and prescription of the user. Moreover, the drug ejection device also serves as a personal digital assistant that is additionally provided with an inhaler for ejecting a drug in the form of fine liquid droplets and allowing the user to inhale the drug, and further includes, in order for the user to inhale the drug according to the information of the prescription, an ejection control unit for controlling, based on an inspiratory profile of the user, the inhaler to eject the drug.

Such a drug ejection device can accurately manage the dose and frequency of administration of the drug according to the prescription, performs appropriate ejection control in accordance with the inspiratory profile of an individual user, and can effectively administer the drug. According to such a drug ejection device, since it is not required to use a conventional medical instrument such as an injection syringe when administering the drug, it is possible not only to easily operate the device without expert knowledge, but also to diminish the pain by a needle of an injection syringe of a user (see WO95/01137 and WO02/04043).

To take insulin as an example, an amount of insulin necessary to be taken by a diabetic patient must be determined according to physical condition of the patient when the patient is going to inhale the drug. In relation to this, the blood glucose level of a healthy person is about 80 to 100 mg/dl in the fasting state and about 150 mg/dl 2 hours after eating. When the blood glucose level increases up to 170 to 180 mg/dl or more, the glucose begins to spill into the urine. According to the above-mentioned drug ejection device, however, it is only possible to preset and control an ejection amount of insulin merely based on a general amount which is determined according to a doctor's prescription.

Also, there may be a case where an individual user adjusts, before eating, an intake amount of insulin at his discretion in consideration of an energy amount that the user is going to ingest and to an amount of exercise that has been performed until then.

The intake amount of insulin needs to be adjusted for the following reasons. That is, carbohydrate and sugar ingested at each meal are absorbed by the intestine and converted into glucose to enter the blood, and transported through the bloodstream to the cells in the body, where the glucose is converted into energy which is to be consumed in muscles and organs. At this time, when insulin is deficient, the glucose courses through the blood without being converted into energy in muscles and organs, while excessive carbohydrate and sugar contained in ingested meal are still converted successively into glucose at the intestine. As a result, the blood glucose concentration increases. On the other hand, when a large amount of energy is consumed through exercise, an amount of glucose (blood glucose) consumed in muscles increases, with the result that the blood glucose level in the blood decreases.

Therefore, an individual user may inhale insulin in an inappropriate amount with respect to physical condition of the user, which results in a high blood glucose level or a low blood glucose level in the blood. Both the high blood glucose level and the low blood glucose level in the blood are detrimental to health. As is generally known, persisting high blood glucose levels do damage to blood vessels, leading to complications associated with organ disorders, which causes cerebral infarction, cerebral embolism, nephropathy, cardiac infarction, retinopathy, arteriosclerosis, neurological disorder, etc. In the case of a low blood glucose, coma or convulsion may be caused, which may lead to death without prompt treatment.

In order to prevent the above-mentioned situations, there has been a method of determining an ejection amount of insulin based on a blood glucose level that is actually measured before meal. However, in order to measure the blood glucose level, it is necessary to take a blood sample by needle penetration through the skin of a user, which causes the user to experience pain and is unacceptable to the user. Alternatively, there has been devised a minimally invasive blood glucose self-monitoring device which is merely necessary to be kept in contact with a human skin surface. However, since the device has a problem in terms of accuracy, and therefore since it is necessary to perform blood sampling and testing periodically (for example, once a day) for correction, the instrument has not been widely accepted by users so far.

For the above-mentioned reasons, the fact is that an ejection amount of insulin from an inhalation device may not be adjusted to be sufficiently appropriate with respect to the physical condition of an individual user or it may be troublesome for users to adjust the ejection amount.

### DISCLOSURE OF THE INVENTION

The present invention has been made to solve the above-mentioned problems and provides an ingesta administration device that can determine an appropriate administration amount of ingesta, without imposing physical strain on a user, while reducing the inconvenience of adjustment.

An ingesta administration device for administering ingesta to a user through an ejection orifice of an ejection portion having the ejection orifice and an ejection energy generating element for ejecting the ingesta according to the present invention includes:

a consumed energy amount obtaining portion for obtaining information on an amount of energy consumed by a user;

an intake energy amount obtaining portion for obtaining information on an amount of energy to be ingested by the user;

a determination portion for determining a administration amount of the ingesta based on at least the information obtained on the amount of energy consumed by the user and the information obtained on the amount of energy to be ingested by the user,

wherein the ejection energy generating element is driven so as to eject the ingesta in an amount corresponding to the administration amount determined by the determination portion.

An ingesta administration method of administrating ingesta to a user according to the present invention includes:

obtaining information on an amount of energy consumed by a user;

obtaining information on an amount of energy to be ingested by the user;

determining a administration amount of the ingesta based on at least the information obtained on the amount of energy consumed by the user and the information obtained on the amount of energy to be ingested by the user; and

driving an ejection energy generating element to eject the ingesta through an ejection orifice in an amount corresponding to the determined administration amount.

The present invention includes the following features. That is, the present invention includes a consumed energy amount obtaining portion, an intake energy amount obtaining portion, a determination portion for determining an administration amount of ingesta, and an ejection portion for ejecting the ingesta according to the administration amount thus determined by the determination portion. The consumed energy amount obtaining portion obtains information on a consumed energy amount based on information on an exercise amount of a user. The intake energy amount obtaining portion obtains information on an amount of energy to be ingested by the user. The term “intake energy amount” herein employed refers to an amount of energy to be ingested by a user by eating and drinking, independently of the ingesta described above. The determination portion determines a administration amount of the ingesta based on at least information obtained by the consumed energy amount obtaining portion on an amount of energy which has been consumed since the previous administration until the current administration, and on information obtained by the intake energy amount obtaining portion on an amount of energy to be ingested by the user from now.

According to the ingesta administration device, such as a drug ejection device, of the present invention, a administration amount of ingesta is determined in consideration of at least information on an amount of energy which has been consumed since the previous administration of the ingesta, and information on an amount of energy to be ingested after the current administration, whereby the ingesta can be administered appropriately corresponding to the physical condition of a user. For example, it is relatively easy to estimate or calculate a necessary amount of insulin which should be inhaled by an individual user, and the inhalation device main body ejects insulin in an appropriate ejection amount corresponding to the necessary amount of insulin thus calculated, whereby a user can perform inhalation with ease. As a result, a blood glucose level in the blood of the user can be maintained in the right conditions, thereby maintaining and controlling the health condition of a user who needs insulin administration.

Other features and advantages of the present invention will be apparent from the following description taken in conjunction with the accompanying drawings, in which like reference characters designate the same or similar parts throughout the figures thereof.

### BRIEF DESCRIPTION OF THE DRAWINGS

FIG. 1 is a partially cut away front view of an inhalation device in a state where an access cover thereof is opened, according to Example 1 of the present invention.

FIG. 2 is a front view of the inhalation device in a state where an access cover thereof is closed, according to Example 1 of the present invention.

FIG. 3 is a block diagram illustrating units for executing respective functions according to an example of the present invention.

FIG. 4 is a flow chart illustrating an operation according to an example of the present invention.

FIG. 5 is a flow chart illustrating an operation of an exercise amount measuring portion according to an example of the present invention.

FIG. 6 is a partially cut away front view of an inhalation device in a state where an access cover thereof is opened, according to Example 2 of the present invention.

FIG. 7 is a partially cut away front view of the inhalation device in a state where an access cover thereof is opened, according to Example 3 of the present invention.

### BEST MODE FOR CARRYING OUT THE INVENTION

Preferred embodiments of the present invention will now be described in detail in accordance with the accompanying drawings.

Examples of ingesta that can be administered by the ingesta administration device of the present invention preferably include ingesta for which a administration amount can be determined physiologically or medically, based on a consumed energy amount and an intake energy amount of a user. Specifically, a drug such as insulin is preferably used. The ingesta may be in the form of liquid or powder, and may include components other than the drug component, such as a stabilizer for preventing deterioration of the drug component.

The consumed energy amount obtaining portion employed in the present invention may be any kind as long as it can obtain information on a consumed energy amount of a user. The consumed energy amount obtaining portion includes an input portion for inputting information on a consumed energy amount and a memory for storing information on a consumed energy amount. The consumed energy amount of a user may be an amount of energy itself consumed by the user, or may be any parameter corresponding to the consumed energy amount.

More preferably, the consumed energy amount obtaining portion may be an exercise amount measuring portion for measuring an exercise amount of a user. It is further desirable that the consumed energy amount obtaining portion include a calculation portion for converting a physical amount or an exercise amount observed by the exercise amount measuring portion into a consumed energy amount or calculating the consumed energy amount based on the physical amount or the exercise amount.

The exercise amount measuring portion is, for example, a measuring instrument which is attached to a user so as to measure an exercise amount of the user, and has an acceleration sensor capable of independently measuring biaxial or triaxial accelerations, the axes being orthogonal to one another. The exercise amount measuring portion can be configured as being attachable to and detachable from the device. Alternatively, in place of the acceleration sensor, a measuring instrument such as a vibration detecting sensor for detecting vibration may also be employed.

The consumed energy amount obtaining portion may also be configured by including an input portion through which a user inputs information on an exercise amount of the user and a calculation portion for calculating a consumed energy amount based on the information on the exercise amount obtained through the input portion. The consumed energy amount obtaining portion may preferably include a calculation portion for calculating a consumed energy amount, not only based on the information on the exercise amount of the user but also by giving consideration to basal metabolism of the user.

The intake energy amount obtaining portion employed in the present invention may be any kind as long as it can obtain information on an amount of energy to be ingested by a user. The intake energy amount obtaining portion includes an input portion through which information on an intake energy amount is input and a memory for storing information on an intake energy amount. The amount of energy to be ingested by a user may be an amount of energy itself to be ingested by the user in the future, or may be any parameter corresponding to the intake energy amount.

The intake energy amount obtaining portion includes an input portion through which a user inputs information on items to be ingested by the user and a calculation portion for calculating an intake energy amount based on the input. In this case, the information on items to be ingested by the user may include an ID number or image data which can specify the ingesta.

The determination portion employed in the present invention determines an administration amount of the ingesta based on at least the information obtained on the consumed energy amount of the user and the information obtained on the amount of energy to be ingested by the user.

It is desirable that the determination portion determine an administration amount of ingesta such as insulin based on at least information obtained by the consumed energy amount obtaining portion on an amount of energy which has been consumed since the previous administration until the current administration, and on information obtained by the intake energy amount obtaining portion on an amount of energy to be ingested by the user after the current administration.

The ingesta ejection portion employed in the present invention has an ejection orifice and an ejection energy generating element for ejecting ingesta through the ejection orifice. The ejection energy generating element may include an electrothermal converter or an electromechanical converter. Specific examples of the electrothermal converter include a resistive heating element, which utilizes thermal energy generated by the resistive heating element to generate an ejection pressure, to thereby eject a liquid containing the ingesta through the ejection orifice as liquid droplets. A specific example of the electromechanical converter includes a piezoelectric element, which utilizes a deformation of the piezoelectric element to generate an ejection pressure, to thereby eject a liquid containing the ingesta through the ejection orifice as liquid droplets.

Then, the drive portion employed in the present invention drives the ejection energy generating element so as to eject the ingesta in an amount corresponding to a administration amount determined by the determination portion. It is also desirable to prohibit the driving of the ejection energy generating element after the amount of ingesta that has been ejected has reached the administration amount determined by the determination portion. For this purpose, specifically, the power supply for the device main body or to the ejection portion may be turned off, or a control signal for suspending the driving of the ejection portion may be transmitted to the ejection portion.

Alternatively, the ejection orifice may be electromechanically covered with a cap after the amount of ingesta that has been ejected from the ejection orifice has reached the administration amount determined by the determination portion, to thereby prohibit the ejection of the ingesta.

An inhalation device as an embodiment of the ingesta administration device of the present invention is an inhalation device which is carried by a user and is used by the user to inhale insulin as a drug. The inhalation device has a consumed energy amount obtaining portion, an intake energy amount obtaining portion, a determination portion for determining an administration amount of insulin, a storage portion for storing insulin, a liquid ejection portion for ejecting insulin as liquid droplets, and a drive portion for driving the liquid ejection portion.

With the above-mentioned structure, it is possible to estimate or calculate, through a relatively simple operation, a necessary amount of insulin which should be inhaled by an individual user, and the inhalation device main body ejects insulin in an ejection amount appropriate for the physical condition of the user corresponding to the thus calculated necessary amount of insulin, whereby the user can perform inhalation with ease. That is, an appropriate administration amount of insulin is determined by estimating a blood glucose level of each user at the time of insulin inhalation based on an exercise amount or the like of the user, and the inhalation device ejects insulin in an appropriate amount, thereby allowing a user to inhale insulin through a simple inhalation operation. In addition, no physical strain is imposed on a user when determining an administration amount of insulin.

Next, the present invention is described in detail based on examples thereof with reference to the accompanying drawings.

### EXAMPLE 1

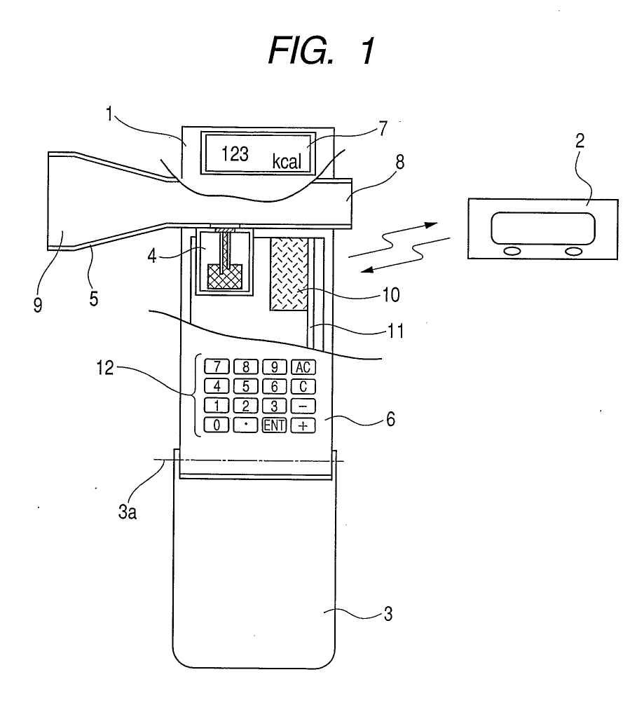

FIG. 1 is a partially cut away front view of an inhalation device according to Example 1 of the ingesta administration device of the present invention. FIG. 1 illustrates a state where an access cover 3 of the device is opened, which is not in an actual usage state. The access cover 3 is coupled to a cover case 6 through a hinge shaft (extending in a direction indicated by dashed line 3a) so as to be opened and closed.

In FIG. 1, the inhalation device includes an inhalation device main body 1 and a three-dimensional acceleration sensor 2 constituting an exercise amount measuring portion which is a part of a consumed energy amount obtaining portion. It is desirable that the three-dimensional acceleration sensor 2 be configured such that the sensor can be put on the body of a user without disturbing the movement of the user so as to measure an exercise amount of the user. In place of the three-dimensional acceleration sensor, a pedometer and a two-dimensional acceleration sensor may be used in combination to thereby attain the same function. It is desirable that the three-dimensional acceleration sensor 2 be separable from the inhalation device main body 1. In this case, the three-dimensional acceleration sensor has a function of exchanging data on an exercise amount with the inhalation device main body 1 through wireless communication (by using Bluetooth, infrared light, or the like). As the acceleration sensor, a well known acceleration sensor such as disclosed in, for example, Japanese Patent Application Laid-Open No. 11-004220 can be used.

Disposed on an upper portion of the inhalation device main body 1 which can easily be viewed by a user is a display portion 7 for displaying time, a message, an input value, etc. for the user. The inhalation device of the present invention is used in a state where a drug ejection unit 4 serving as an ejection portion, which includes a tank (a storage portion) for storing insulin and an ejection head (a liquid droplet ejection portion) and a mouthpiece 5 which is used by a user for inhalation are mounted on the main body 1. Arranged within the inhalation device main body 1 is a wireless communication unit 10 which is disposed on a control board 11. Further, arranged at a lower portion of the surface of the inhalation device main body 1 is an input key 12 serving as an input portion, which is used to input information relating to an intake energy amount and personal information such as body weight, body height, body temperature, and blood pressure.

An inhalation flow path for drug inhalation is defined by an inner wall of the mouthpiece 5. The mouthpiece is made attachable to and detachable from the main body in order to prevent an inner portion of the main body from being contaminated. The mouthpiece 5 has an air inlet port 8 for the inhalation flow path and an inhalation port 9.

When a user performs an inhalation operation, the drug ejection unit 4 ejects liquid droplets through the ejection head. Then, insulin is carried by a flow of airs from the air inlet portion 8 to the inhalation port 9 generated by the inhalation of the user, and enters, in the form of liquid droplets, the lung of the user through the inhalation flow path of the mouthpiece 5. The insulin that has entered the lung of the user is absorbed by the lung alveoli to be transported throughout the body via the blood vessels, so as to help an internal respiration action of cells for converting glucose into energy.

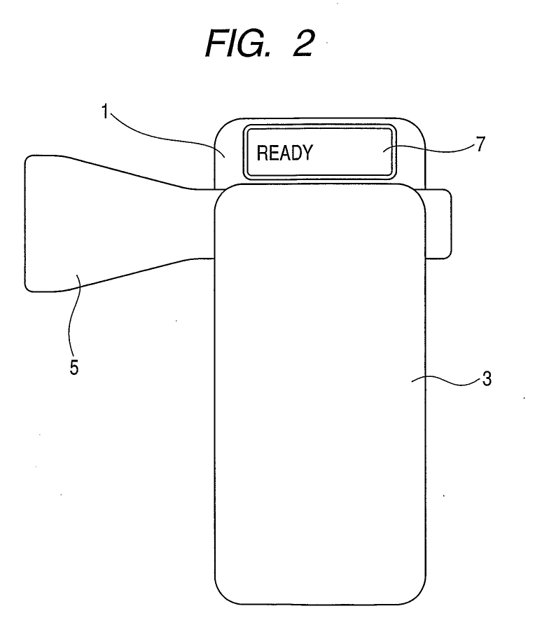

FIG. 2 is a front view of the inhalation device in a state where the access cover 3 is closed. When the administration amount of insulin has been determined by the determination portion, a message “READY” is displayed on the display portion 7 so as to indicate that the device is ready for inhalation. The user confirms the display and performs inhalation.

According to the above-mentioned structure, the drug ejection unit 4 and the mouthpiece 5 are provided independently of each other. However, in view of the fact that insulin is a protein formulation, it is desirable, from the viewpoint of hygiene, to make the drug ejection unit 4 and the mouthpiece 5 disposable or to periodically replace the drug ejection unit 4 and the mouthpiece 5. Therefore, the drug ejection unit 4 and the mouthpiece 5 may be integrated into a single body.

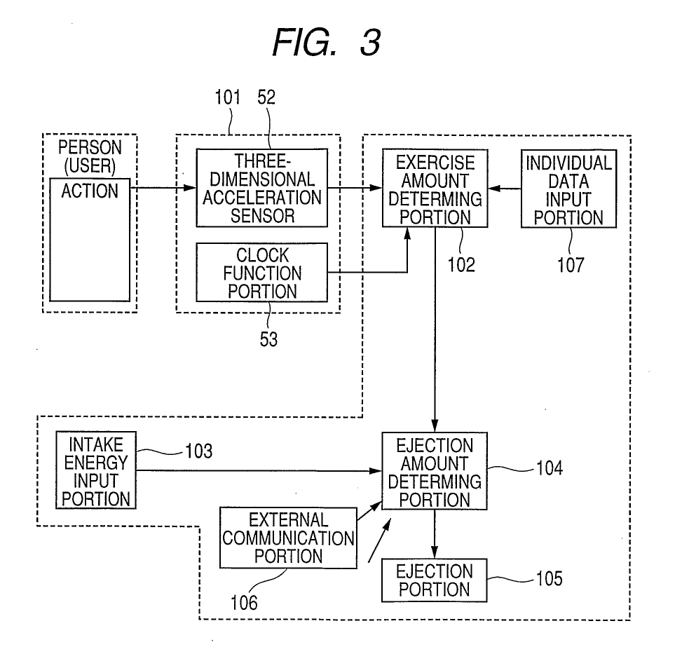

FIG. 3 is a block diagram of a general basic structure illustrating a connection relation between units for executing respective functions of this example. The inhalation device includes a consumed exercise amount obtaining portion which includes an exercise amount measuring portion 101 (corresponding to reference numeral 2 of FIG. 1) and an exercise amount determining portion 102, an intake energy amount input portion 103 of an intake energy amount obtaining portion, an insulin ejection amount determining portion 104 of a determination portion, an ejection portion 105 (corresponding to reference numeral 4 of FIG. 1), and an external communication portion 106.

The exercise amount measuring portion 101, in FIG. 3, uses a three-dimensional acceleration meter 52, a clock function portion 53, and the like to obtain data, and calculates a consumed energy amount, which has been consumed by a user since the previous inhalation, by the exercise amount determining portion 102 serving as a calculation portion, based on the data thus obtained while giving consideration to basal metabolism which varies between individuals, the basal metabolism being determined based on data such as gender, body height, body weight, muscular strength which is input by an individual data input portion 107. The consumed energy amount is expressed by a product of an exercise intensity and exercise time. The exercise intensity is a value obtained by adding the amount of metabolism at rest to a product of the energy metabolic rate and the amount of basal metabolism. The energy metabolic rate indicates how many times of the basal metabolic amount are consumed depending on forms of exercise. For example, an energy amount consumed by stroll is two times, an energy amount consumed by walking is three times, an energy amount consumed by trotting is five times, and an energy amount consumed by running is seven times, and therefore the energy amount can be obtained through actual measurement. The calculation is performed on the assumption that the amount of metabolism at rest is 1.2 times the basal metabolism amount, which is multiplied by the exercise time, and the consumed energy can be obtained by adding all the amounts.

Further, the intake energy input portion 103 is used to input information on an amount of energy to be ingested. The ejection amount determining portion 104 serving as the determination portion described above estimates a blood glucose level based on an amount of energy which has been consumed since the previous inhalation and an amount of energy to be ingested from now, and calculates how much amount of insulin needs to be administered in order to maintain a blood glucose level which is necessary to the body.

The external communication portion 106 communicates with a healthcare center which supports a user, through a communication unit that is commonly used at present (wireless communication unit such as a mobile phone and a PHS), and is used to receive data on energy of new items of food and drink and the like. Also, the external communication portion is capable of transmitting data necessary for determining an administration amount and obtaining data on the administration amount. The drug ejection unit 4 ejects liquid droplets of insulin in a predetermined amount when a user performs inhalation, based on a calculated value obtained by the ejection amount determining portion 104.

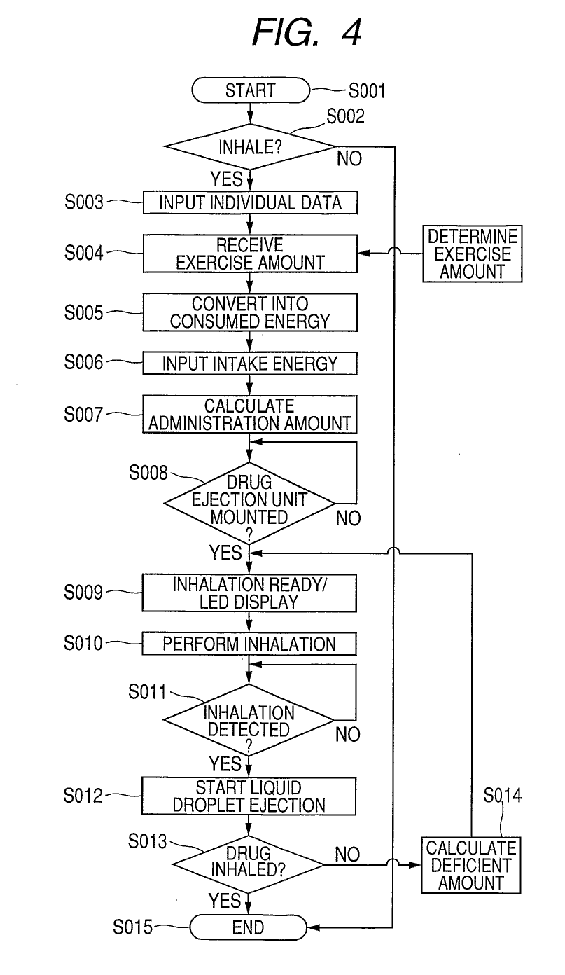

FIG. 4 is a flow chart of an operation of this example. With reference to the flow chart, a basic operation of this example is described.

First, a user performs an operation, for example, by pressing a power switch to get the device ready to use (starting step S001). Next, in response to a question asked by this inhalation device whether the user performs inhalation or not (S002), when the user answers “YES”, the device is put into a mode in which personal data can be input (S003). When the user answers “NO”, the operation immediately ends (S015). As the personal data of the user, body height, body weight, and the like, are input, and the device grasps the basal metabolism of the user based on the data. It is needless to add that, when the user already knows the basal metabolism of him, the value of the basal metabolism may directly be input. Also, in a case where the device is used by a specific user every time, it is necessary to input personal data just once when the user uses the device for the first time, and the input of the personal data can be omitted from the next time.

Next, the device is put into a mode for obtaining an exercise amount. The exercise amount measuring portion 2 may be integrated with the inhalation device main body 1, or may be separate therefrom. When the exercise amount measuring portion 2 is separate from the inhalation device main body 1, since the exercise amount measuring portion 2 is put on the body of a user, the exercise amount measuring portion 2 observes an exercise amount gained after the previous inhalation and stores an acceleration in the moving direction of the user, an acceleration in the vertical direction, acceleration intensities thereof, and the like in an internal memory, and in response to a transmission command from the inhalation device main body 1, the exercise amount measuring portion 2 transmits the data on exercise amount (S004). Based on the data on exercise amount thus transmitted, the calculation unit (the exercise amount determining portion 102), which includes a CPU, a RAM, a ROM, etc., disposed on the control board 11 of the main device, classifies the data into respective exercise patterns (such as level ground walking, level ground running, and stairs up/down). Then, the exercise pattern, the exercise intensity, and the exercise amount, are calculated as (i.e., converted into) the amount of energy that has been consumed since the previous inhalation until the current inhalation (S005). In an example where the exercise amount measuring portion 2 is formed integrally with the inhalation device main body 1, data on an exercise amount is input as such into the calculation unit (the exercise amount determining portion 102), and calculated as (i.e., converted into) the consumed energy amount as described above.

Alternatively, there may be adopted a method in which a user inputs the data on exercise amount displayed in the exercise amount measuring portion (including an acceleration sensor, a vibration detecting sensor, or a pedometer as described above) into the device main body 1. In this case, the calculation unit (the exercise amount determining portion 102) calculates the consumed energy amount based on the input.

Next, information on an amount of energy of food and drinks which are to be ingested from now on is input (S006). This may be performed by inputting caloric values indicated by a calorie list of food and drinks which is currently commonly seen, by ten key operation. Alternatively, the input is made by selecting items from a menu list displayed.

Then, based on the intake energy amount thus input or calculated and the consumed energy amount thus calculated, the calculation unit (ejection amount determining portion 104) calculates an amount of insulin necessary to be ingested (S007).

The greater the consumed energy amount thus calculated is, the more the blood glucose level decreases. Therefore, a smaller amount of insulin is necessary to be administered. Further, the greater the intake energy amount thus input or calculated is, the more the blood glucose level after the ingestion will increase. Therefore, a greater amount of insulin is necessary to be administered. Based on the values of consumed energy and intake energy, the administration amount of insulin is calculated.

In order to calculate the administration amount of insulin, a reduction rate of the blood glucose level with respect to the consumed energy amount of a user and an increasing rate of the blood glucose level with respect to the intake energy amount of the user are necessary, because individual users respond to insulin differently depending on their own basal metabolisms which are different according to their physiques, constitutions, etc. Therefore, in order to calculate an administration amount suitable to an individual user, it is desirable to store, in the calculation unit, rates of reduction/increase measured based on an amount of change (reduction) in blood glucose level when a predetermined amount of exercise load is given and an amount of change (increase) in blood glucose level when a predetermined amount of energy is ingested. In this manner, the amount of insulin to be administered is calculated such that the blood glucose level, which temporarily increases after eating, then decreases to about 150 mg/dl 2 hours after eating, and further decreases to about 80 to 100 mg/dl in the fasting state before the subsequent eating. A predicted amount of exercise to be performed after eating may be input when determining the current administration amount so as to be reflected on the current administration amount. In a case where the actual amount of exercise performed by the user after the current administration of insulin and meal exceeds the predicted amount of exercise, and there is a fear that the blood glucose level decreases to be too low, a signal (such as sound, vibration, or light) may be output to urge the user to replenish the glucose level. In addition, it is also possible to calculate an administration amount suitable to an individual user while giving consideration to a lifestyle pattern, an occupation, and the like, which are related to the consumed energy amount, of the user.

When the administration amount of insulin is determined, it is checked whether the drug ejection unit 4, which is made integrally with a tank for storing insulin, is mounted on the device main body 1 or not (S008). When the drug ejection unit 4 performs ejection through a thermal ink jet system, the detection of the presence/absence of the drug ejection unit 4 can be implemented by measuring a resistance value of a resistive heating element (heater) serving as an ejection energy generating element. It is also possible, of course, to adopt another system, such as a piezoelectric element system, as a system for the ejection unit.

When the drug ejection unit 4 is not detected, a display for urging the user to re-attach the drug ejection unit 4 is displayed on the display portion 7, so as to inform the user. Then, the presence/absence of the drug ejection unit 4 is checked again. On the other hand, when the drug ejection unit 4 is detected, a display of “inhalation READY” is displayed on the display portion 7 (S009). The display may also be displayed through an LED or the like.

When the user sees the signal indicating that the inhalation preparation is completed, the user starts an inhalation operation (S010). When the inhalation is detected (S011), a display for notifying the user that the ejection is being performed is displayed and a drug is ejected from the ejection unit 4 (S012). In order to detect inhalation, it is possible to use a sensor capable of measuring an air flow, such as a negative pressure sensor or a flowmeter. The sensor is provided to a flow path formed in the mouthpiece 5 in such a manner that the sensor is in communication with the flow path.

Next, it is checked whether a predetermined amount has been inhaled (S013). Since the amount of inhalation performed by a user can be calculated based on time integration of values detected with the above-mentioned negative pressure sensor or the like, the amount of insulin inhaled can be detected. When the amount of insulin inhaled through a single inhalation operation is less than the determined administration amount, the user is urged to perform re-inhalation, while a deficient amount is calculated by the calculation unit (S014), and the process returns to the step of “inhalation READY” (S009). In this manner, when the user, who is urged to perform re-inhalation, performs re-inhalation until an appropriate amount is inhaled, the power is turned off to end the process (S015).

When a determined administration amount is inhaled through a single inhalation operation, the ejection portion 4 is controlled to stop the ejection after the determined administration amount has been ejected.

Also, even after the ejection is completed, it is not necessary to turn off the power immediately, as long as a current supply to the head is shut off so as to make the ejection impossible.

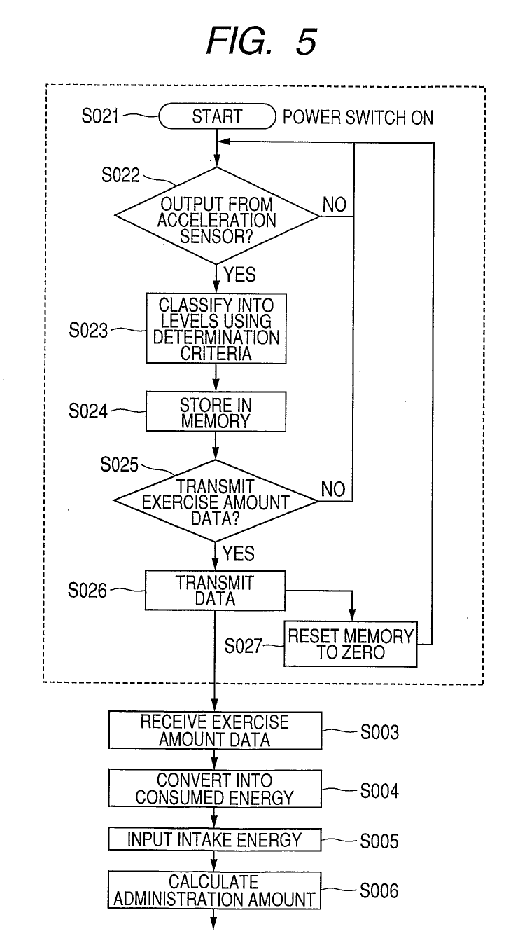

Next, with reference to FIG. 5, a flow chart in a case of using the divided-type exercise amount measuring portion 2 is described in detail. The power switch of the exercise amount measuring portion 2 is turned on so as to start measuring at the three-dimensional acceleration sensor 52 (S021). When a signal is output from the three-dimensional acceleration sensor (S022), the output is divided into different levels (for example, roughly into a first, second, and third levels) with reference to a determination criteria stored in an internal memory (S023), and the measured data and/or the level thereof obtained from the acceleration sensor are/is recorded/stored in a flash memory or the like in the measuring portion (S024). After that, in response to a transmission command from the inhalation main body 1 (S025), the data on an exercise amount thus accumulated is transmitted to the inhalation main body 1 (S026), and the data is converted into the consumed energy amount so as to be used as a parameter for calculating an administration amount. At this time, simultaneously with the transmission of the data on an exercise amount to the inhalation device main body 1, the memory of the exercise amount measuring portion 2 is reset to zero, to thereby initialize measured data on an exercise amount (S027). By the initialization, it is possible to obtain information on the consumed energy amount of a user, which has been consumed since the current ingesta administration until the next time ingesta administration. The subsequent process is the same as described with reference to the flow chart of FIG. 4.

### EXAMPLE 2

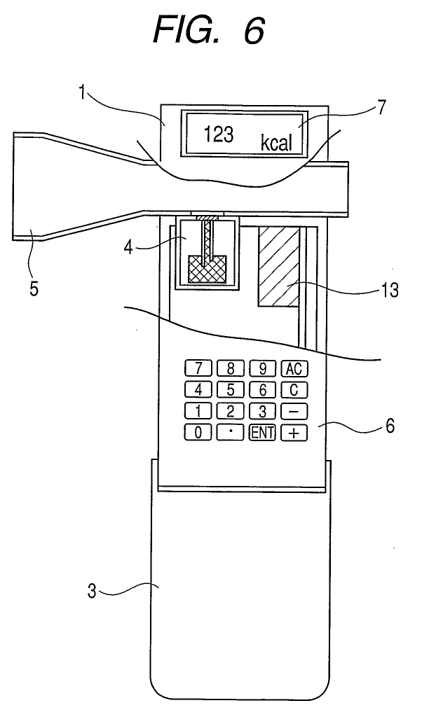

FIG. 6 is a partially cut away front view of an inhalation device according to Example 2 of the ingesta administration device of the present invention. According to this example, a biaxial or three-dimensional acceleration sensor 13 for measuring an exercise amount, which is provided independently of the inhalation device main body 1 according to Example 1, is integrated into the inhalation main body 1. Unlike Example 1, it is unnecessary to provide a communication function, so that the size and weight of the device can be reduced correspondingly. Further, the production cost can also be reduced. For the rest, Example 2 is similar to Example 1.

### EXAMPLE 3

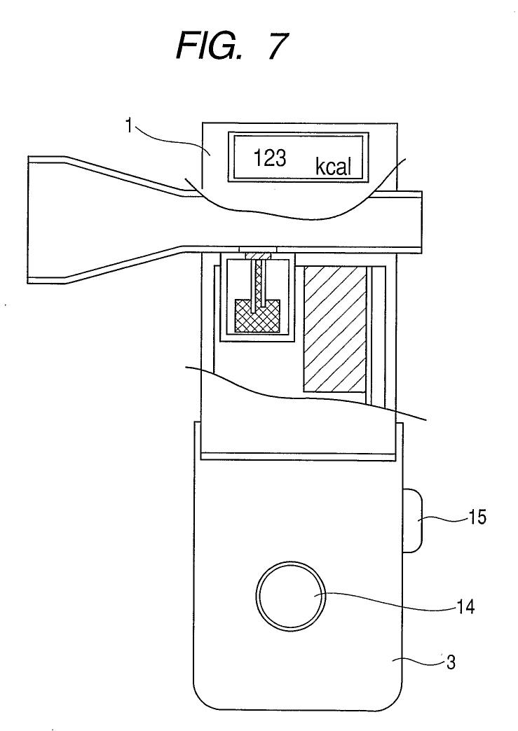

FIG. 7 illustrates Example 3 in which a function of a digital camera is used as a data input portion of the intake energy amount obtaining portion. With the development of a mobile phone, a digital camera function has been reduced in size and can be attained at low cost, which makes it easy to install the function into a mobile terminal device. According to this example, a digital camera serving as an input portion is provided to a portion of the access cover 3 of the inhalation main body 1, so as to serve as an input portion. With this structure, a lens unit 14 is directed toward an item of food and drink to capture an image thereof, and adjust the image into an appropriate size so as to make it easy to perform image processing thereon. The adjustment made on the image size is confirmed on a monitor (not shown). Then, a shutter button 15 is pressed so as to capture image data of the object by an internal imaging taking element through the lens unit 14, and the image data is extracted after being subjected to image processing in terms of color and shape. Based on the image data, the calculation portion compares the extracted image with image data of food and drink stored in the inhalation device main body 1, to thereby specify the item of food and drink and calculate the intake energy amount of the item of food and drink.

Unlike Example 1 and Example 2, in which a user inputs, by key operation, information for calculating the intake energy amount, according to this example, an item of food and drink is determined through image processing so as to calculate the intake energy amount. Accordingly, the trouble of key input can be avoided while eliminating the need to provide an input key to the inhalation device main body 1 at the same time. In this manner, the device is made easy to operate for a user while being further reduced in size, which increases portability. For the rest, this example is similar to Example 1.

In the above-mentioned examples, insulin is taken as an example of the ingesta, but the ingesta that can be used in the administration device of the present invention are not limited to insulin but various kinds of ingesta (such as a drug, a nutritional supplement, and nicotine) can be used as the ingesta. For example, in a case where a patient with both diabetes and another disease, the ejection unit may be replaced with an ejection unit that includes another drug in addition to the above-mentioned insulin to use the device.

The present invention is not limited to the above embodiments and various changes and modifications can be made within the spirit and scope of the present invention. Therefore to apprise the public of the scope of the present invention, the following claims are made.

This application claims priority from Japanese Patent Application No. 2006-107138 filed on Apr. 10, 2006, which is incorporated hereinto by reference.

1. An ingesta administration device for administering ingesta to a user through an ejection orifice of an ejection portion having the ejection orifice and an ejection energy generating element for ejecting the ingesta, which comprises:
a consumed energy amount obtaining portion for obtaining information on an amount of energy consumed by a user;
an intake energy amount obtaining portion for obtaining information on an amount of energy to be ingested by the user; and
a determination portion for determining a administration amount of the ingesta based on at least the information obtained on the amount of energy consumed by the user and the information obtained on the amount of energy to be ingested by the user,
wherein the ejection energy generating element is driven so as to eject the ingesta in an amount corresponding to the administration amount determined by the determination portion.

2. The ingesta administration device according to claim 1, wherein the determination portion determines the administration amount of the ingesta based on information on an amount of energy consumed by the user since the previous administration of the ingesta until the current administration of the ingesta and information on an amount of energy to be ingested by the user after the current administration.

3. The ingesta administration device according to claim 1, wherein the consumed energy amount obtaining portion comprises an exercise amount measuring portion for measuring an exercise amount of the user.

4. The ingesta administration device according to claim 3, wherein the exercise amount measuring portion is attachable to and detachable from a body of the device.

5. The ingesta administration device according to claim 1, wherein the consumed energy amount obtaining portion comprises an input portion through which the user inputs information on an exercise amount.

6. The ingesta administration device according to claim 3, wherein the consumed energy amount obtaining portion comprises a calculation portion for calculating a consumed energy amount based on an information on the exercise amount obtained through one of the exercise amount measuring portion and the input portion.

7. The ingesta administration device according to claim 6, wherein the calculation portion calculates the consumed energy amount based on the information on the exercise amount obtained through one of the exercise amount measuring portion and the input portion while giving consideration to basal metabolism of the user.

8. The ingesta administration device according to claim 1, wherein the intake energy amount obtaining portion comprises an input portion through which the user inputs information on an item to be ingested and a calculation portion for calculating an intake energy amount based on the input information.

9. The ingesta administration device according to claim 1, wherein the ingesta comprises insulin.

10. The ingesta administration device according to claim 1, wherein the ejection energy generating element is prohibited from being driven after ejecting the ingesta in an amount corresponding to the administration amount determined by the determination portion.

11. An ingesta administration method of administrating ingesta to a user comprising:
obtaining information on an amount of energy consumed by a user;
obtaining information on an amount of energy to be ingested by the user;
determining a administration amount of the ingesta based on at least the information obtained on the amount of energy consumed by the user and the information obtained on the amount of energy to be ingested by the user; and
driving an ejection energy generating element to eject the ingesta through an ejection orifice in an amount corresponding to the determined administration amount.

12. The ingesta administration method according to claim 11, wherein the administration amount of the ingesta is determined based on information on an amount of energy consumed by the user since the previous administration of the ingesta until the current administration of the ingesta and information on an amount of energy to be ingested after the current administration.

13. The ingesta administration device according to claim 4, wherein the determination portion determines the administration amount of the ingesta based on information on an amount of energy consumed by the user since the previous administration of the ingesta until the current administration of the ingesta and information on an amount of energy to be ingested by the user after the current administration.

14. The ingesta administration device according to claim 2, wherein the consumed energy amount obtaining portion comprises an exercise amount measuring portion for measuring an exercise amount of the user.

15. The ingesta administration device according to claim 3, wherein the consumed energy amount obtaining portion comprises a calculation portion for calculating a consumed energy amount based on an information on the exercise amount obtained through one of the exercise amount measuring portion and the input portion.

16. The ingesta administration device according to claim 14, wherein the consumed energy amount obtaining portion comprises a calculation portion for calculating a consumed energy amount based on an information on the exercise amount obtained through one of the exercise amount measuring portion and the input portion.

17. The ingesta administration device according to claim 5, wherein the consumed energy amount obtaining portion comprises a calculation portion for calculating a consumed energy amount based on an information on the exercise amount obtained through one of the exercise amount measuring portion and the input portion.
